(19) United States
(12) Patent Application Publication
(10) Pub. No.: US 2009/0203944 A1
(43) Pub. Date: Aug. 13, 2009

(54) Oxidation Catalyst

(76) Inventors: Zengjian An, Dalian (CN); Xinhe Bao, Dalian (CN); Xiuwen Han, Dalian (CN); Xiumei Liu, Dalian (CN); Xiulian Pan, Dalian (CN)

Correspondence Address: NIXON & VANDERHYE, PC, 901 NORTH GLEBE ROAD, 11TH FLOOR, ARLINGTON, VA, 22203, US

(21) Appl. No.: 12/309,020

(22) PCT Filed: Jun. 28, 2007
(86) PCT No.: PCT/GB2007/002411

§ 371 Date: Jan. 2, 2009

(30) Foreign Application Priority Data
Jul. 4, 2006 (CN) PCT/CN2006/001564

(51) Int. Cl. C07C 31/04; B01J 31/02; B01J 31/04

(52) U.S. Cl. 568/910; 502/168; 502/173; 502/200; 502/172

(57) ABSTRACT

A catalyst for the oxidation of an alkane to an oxygenated hydrocarbon in the presence of oxygen as a first oxidant, comprising a redox active metal centre that can be present in an oxidised and in a reduced form, an acid, a second oxidant for oxidising the reduced form of the redox active metal centre, and a source of nitrous oxide.

This invention relates to the field of catalysis, more specifically to a catalyst for the direct oxidation of methane to oxygenated hydrocarbons in the presence of oxygen.

Converting natural gas to oxygenated hydrocarbons is typically achieved industrially in two stages. First, the methane is converted to syngas (a mixture of carbon monoxide and hydrogen) by processes such as partial oxidation, steam reforming or autothermal reforming. The second stage is the conversion of the syngas into oxygenated hydrocarbons, for example the production of methanol using a Cu/ZnO/Al₂O₃ catalyst, or the production of ethanol and/or higher hydrocarbons using a rhodium catalyst.

In order to minimise the complexity of the process, the direct conversion of methane into oxygenated hydrocarbons using a single stage would be of a considerable advantage.

WO 92/14738 describes a process for reacting methane with a strong acid in the presence of a metallic catalyst and an oxidising agent. The product is the methyl salt or ester of the acid. The examples of WO 92/14738 include catalytic systems comprising palladium as the active metal, triflic acid or sulphuric acid as the acid, and oxygen as the oxidising agent.

Although oxygen is a desirable oxidant to use, due to its low cost and high abundance, the methane conversions achieved when it is used tend to be low. Other oxidants, such as SO₃, persulphate or peracids, can improve conversions, but they are relatively expensive and constantly need to be replaced in order to maintain the catalytic reaction. WO 92/14738 describes how a mercury catalyst, in the presence of sulphuric acid, is able to oxidise methane more effectively than other metals, such as palladium, thallium, gold and platinum, in the presence of oxygen, and optionally in the presence of SO₃. However, as mercury is a toxic and environmentally damaging metal, there remains a need for a catalyst and process for the oxidation of a hydrocarbon with a high oxygenate yield, but which avoids the necessity for such potentially damaging components.

According to the present invention, there is provided a catalyst for the oxidation of a hydrocarbon to an oxygenated hydrocarbon in the presence of oxygen as a first oxidant, which catalyst comprises a redox active metal centre, an acid, and a second oxidant, characterised in that the catalyst additionally comprises a source of nitrous oxide.

The catalyst of the present invention is capable of converting a hydrocarbon to an oxygenated hydrocarbon in the presence of oxygen. When in use, nitrous oxide generated from the source of nitrous oxide provides superior catalytic activity and enhances yield of the oxygenated hydrocarbon compared to a catalyst that is absent the source of nitrous oxide.

The catalyst may be a homogeneous catalyst, in which the components are mixed or dissolved in a liquid phase, for example being dissolved in a liquid acid. Alternatively, the catalyst may be heterogeneous, in which one or more of the components are in the solid phase, for example where the components are supported on a refractory oxide or a solid acid, such as an aluminosilicate zeolite. Homogeneous catalysts are preferred, as they are typically more active than heterogeneous counterparts under milder conditions, and allow improved contact between the constituent components of the catalyst.

Sources of nitrous oxide (NO) include nitrous oxide itself, other oxides of nitrogen such as NO₂, N₂O₃, N₂O₄ and N₂O₅, salts comprising anionic oxides of nitrogen such as NO₂⁻ (nitrite), and salts comprising NO⁺ (nitrosonium) cations. Suitable compounds comprising nitrite ions include alkali metal salts, alkaline earth metal salts and transition metal salts. In one embodiment, the cation of the nitrite salt is the redox active metal centre of the present composition. Suitable compounds comprising nitrosonium ions include nitrosyl salts of tetrafluoroborate ([NO]BF₄) and perchlorate ([NO]ClO₄), and nitrosyl sulphuric acid ([NO]HSO₄). Conveniently, an alkali metal nitrite salt is the source of nitrous oxide, such as sodium or potassium nitrite, which can generate nitrous oxide in the presence of an acid.

The source of nitrous oxide releases or produces nitrous oxide when the catalyst is in use. The nitrous oxide, when the catalyst is in use, is reversibly oxidised to NO₂ in the presence of oxygen, which in turn is able to regenerate the oxidised form of the electron transfer agent that has been reduced during re-oxidation of the reduced redox active metal centre. The use of a source of nitrous oxide in the catalyst of the present invention is advantageous, as the nitrous oxide/nitrogen dioxide cycle is stable under the acidic conditions prevalent when the catalyst is in use, unlike macrocyclic metal complexes such as cobalt-porphyrin complexes.

The catalyst comprises a redox active metal centre which can exist in an oxidised and in a reduced form. In this context, the term “metal” includes those elements described as metalloids, such as germanium, antimony, tellurium and the like. Most transition metals, lanthanides and actinides are capable of existing in more than one form, as are a number of main group metals. Examples of metals suitable for use as the redox active metal in the present invention include Cu, Zn, Pd, Ag, In, Sn, Sb, Te, Pt, Au, Pb, Bi, Ga, Ge, As, Rh, Ir, Os and Ru. Although metals such as Hg, Cd or Tl are also capable of being used in the present invention, they are preferably avoided due to their high toxicity and potential for environmental damage. In a preferred embodiment, the redox active metal is selected from V, Fe, Co, Ni, Cu, Rh, Pd and Pt.

The redox active metal centre can be provided in any form such that, when in use, it is capable of cycling between two oxidation states. Thus, for example, it can be introduced in the metallic (0 oxidation state) form, or as a compound or complex in which the metal centre is in a higher oxidation state. For example, the redox active metal centre can be added to the catalyst as a salt, such as a nitrate, sulphate, oxalate, halide, acetate. In one embodiment, the redox active metal centre can be coordinated to the anion and/or any other ligands, such as amines, phosphines, oximes, or macrocyclic ligands, such as crown ethers, porphyrins, salophens and the like. In another embodiment, it can be added in the form of an oxide. In yet another embodiment it can be provided in a compound having more than one redox active metal centre, such as a heteropolyacid, for example in the form of molybdovanadophosphoric acid having general formula H₃₊ₓPMo₍₁₂₋ₓ₎Vₓ, where x is typically between 1 and 3. In this embodiment, the heteropolyacid can also function as the acid component of the catalyst.

When in use, the redox active metal centre is capable of being present in an oxidised form and a reduced form such that the metal centre can cycle between two different oxidation states, for example Pd(0)/Pd(II), Pt(0)/Pt(II) and/or Pt(II)/Pt(IV), Rh(I)/Rh(III), Ni(0) and Ni(II) and Co(II)/Co(III). In the oxidation of alkanes, such as methane oxidation, the redox active metal centre oxidises, or activates, the hydrocarbon by cleaving a carbon-hydrogen bond. This can be through a homolytic mechanism, via a free-radical pathway, or by a heterolytic mechanism. One-electron redox cycles tend to result in homolytic cleavage of the C—H bond, which produces highly reactive free radicals which can attack or decompose one or more of the catalyst constituents. Therefore, two-electron redox cycles are preferred, which tend to promote heterolytic cleavage of C—H bonds. This prolongs the lifetime of the catalyst components, and improves selectivity to desired products. Preferred redox active metal centres with two-electron redox cycles are Ni, Rh, Pd or Pt.

The redox active metal centre can be associated with a promoter or co-catalyst, which enhances the rate of catalysis and/or improves catalyst lifetime and/or improves product selectivity. In one embodiment, the promoter or co-catalyst is a second redox-active metal centre. In yet a further embodiment of the invention, the second redox active metal centre acts as the second oxidant, and transfers electrons between the first metal centre and the source of nitrous oxide. As an example, Cu can be used as a second oxidant in a catalyst comprising both Pd and Cu, in which Cu(II) species oxidise Pd(0) species to Pd(II), the Cu(II) being reduced to Cu(I) as a result. The Cu(II) is regenerated from Cu(I) by the source of nitrous oxide, which in turn is converted into nitrous oxide.

The catalyst composition comprises an acid. The acid, which can act as a solvent for the other catalyst components in a homogeneous system, is able to form an ester with the oxidised hydrocarbon. In the case of methane oxidation, for example, the acid forms a methyl ester. Examples of acids suitable for use in the present invention are typically strong Brønsted acids, and include inorganic mineral acids, such as heteropolyacids (for example phosphotungstic acid, silicotungstic acid, phosphomolybdic acid, or silicomolybdic acid, or substituted analogues thereof such as molybdovanadophosphoric acid), sulphuric acid, oleum, methyl sulphonic acid, trifluoromethyl sulphonic acid, and organic acids such as trifluoroacetic acid.

In use, during oxidation of the hydrocarbon, the redox active metal centre is reduced to a lower oxidation state. For catalysis to be maintained, the metal centre is reoxidised to a higher oxidation state by a second oxidant. Although oxygen (the first oxidant) is capable under some circumstances of achieving the re-oxidation of the metal centre, the oxidation is typically very slow. The presence of a second oxidant in the catalyst composition is able to enhance the rate of re-oxidation of the metal centre. Examples of second oxidants suitable for use in the present invention include peroxides, such as hydrogen peroxide, tert-butyl hydrogen peroxide or cumene hydroperoxide, a peracid such as peroxyacetic acid, a quinone, quinone derivatives, and a second redox active metal centre. Suitable second redox-active metal centres that can be used as a second oxidant are Cu, Fe or Co, which in one embodiment can be provided in the form of a porphyrin or salophen complex.

When the catalyst is in use, the source of nitrous oxide produces nitrous oxide. Nitrous oxide is oxidised in the presence of oxygen to nitrogen dioxide. The nitrogen dioxide in turn can oxidise the reduced second oxidant, and re-create the nitrous oxide.

An advantage of the present invention is that only catalytic amounts of the catalyst components are required, as opposed to stoichiometric amounts, and only oxygen and the hydrocarbon are consumed in the process.

In a particularly preferred embodiment, the second oxidant is a quinone or a derivative thereof. Quinones and their derivatives tend to be more resistant to deactivation compared to other oxidants, such as transition metal macrocyclic complexes, when the catalyst is in use. Derivatives of quinones comprise the basic quinone unit (i.e. O═C₆H₄═O) with one or more of the carbon atoms having a functional group, such as an alkyl, aryl, halide, hydroxide, ester or ether. When in use, the quinone or quinone derivative oxidises the reduced form of the redox active metal to form hydroquinone. This is achieved in the presence of acid, requiring two protons to balance the negative charges acquired on reduction of the quinone unit. When the hydroquinone is oxidised, the protons are re-released. Before use, the quinone or derivative thereof may be present in the catalyst in the oxidised or reduced form, i.e. as quinone or hydroquinone (or derivative thereof).

The source of nitrous oxide is particularly beneficial when used in conjunction with a quinone or quinone derivative in the catalyst of the present invention. A high degree of reoxidation of the hydroquinone to quinone (or derivatives thereof) can be achieved, which in turn benefits the rate of catalysis and yield of oxygenated hydrocarbon when the catalyst is in use.

Typically, the molar ratio of the redox active metal centre to the second oxidant is in the range of from 1:100 to 100:1, preferably in the range of from 1:0.5 to 1:50. The molar ratio of redox active metal centre to the source of nitrous oxide is suitably in the range of from 1:100 to 100:1, preferably in the range of from 1:0.5 to 1:50.

The catalyst can be used in the oxidation of hydrocarbons to oxygenated hydrocarbons in the presence of oxygen. Oxygenated hydrocarbon products include alcohols, ethers, esters, carboxylic acids, epoxides, aldehydes and ketones. In one embodiment, the catalyst can be used to oxidise an alkane, for example a C₁ to C₄ alkane, to an alcohol. The catalyst shows surprisingly high activity towards the direct oxidation of methane to methanol. Temperatures typically used in methane oxidation reactions are in the range of from 50 to 250° C., and pressures up to 100 barg (10.1 MPa), for example in the range of from 20 to 70 barg (2.1 to 7.1 MPa).

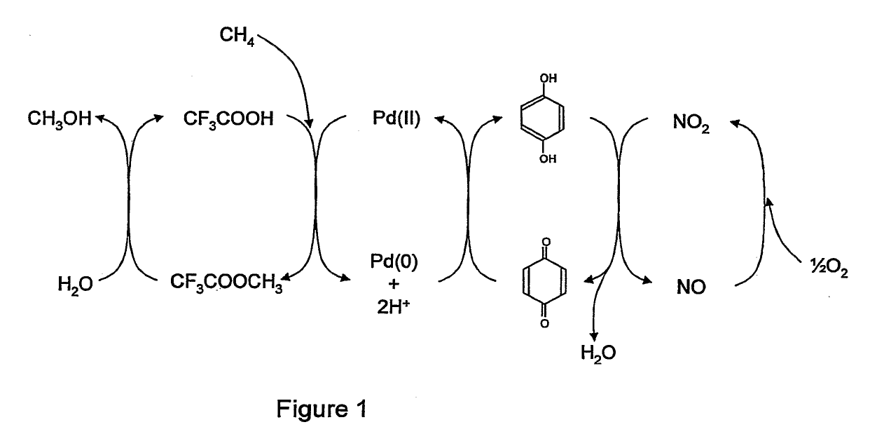

The invention will now be illustrated by the following non-limiting examples and by FIG. 1, which shows a schematic overview of a methane oxidation mechanism using a catalyst in accordance with the present invention;

In FIG. 1, a typical catalytic mechanism is illustrated for a homogeneously catalysed methane oxidation reaction in the presence of oxygen (the first oxidant), in which the redox active metal centre is palladium, the acid is trifluoroacetic acid, the second oxidant is para-quinone, and the source of nitrous oxide is a nitrite salt (in the form of sodium nitrite). In this embodiment, the trifluoroacetic acid, in the presence of a Pd(II) redox active centre, reacts with methane to produce methyl trifluoroacetate and two protons, the palladium being reduced in the process to Pd(0). The Pd(0) is oxidised back to Pd(II) by para-quinone in the presence of the two protons to produce hydroquinone. In turn, the hydroquinone is reoxidised to para-quinone by the action of nitrogen dioxide, which in turn is reduced to nitrous oxide, releasing water. The nitrous oxide is oxidised to nitrogen dioxide by oxygen. Methanol is released from the methyl trifluoroacetate by hydrolysis with water (catalysed by acid). The net result of the process can be expressed by the formula:

CH₄+½O₂→CH₃OH

### EXPERIMENT 1

A 50 mL glass-lined autoclave was charged with a ptfe-coated magnetic stirrer, the desired quantities of palladium acetate and second oxidant, and 3 mL trifluoroacetic acid. The autoclave was purged three times with methane at 30 atm, and then charged with 55 atm methane. The autoclave was then heated in an oil bath held at 80° C. over a period of 10 hours under constant stirring, before being quenched in an ice bath and depressurising the autoclave.

The product identities were determined using GC-MS and NMR spectroscopy, and quantified by GC.

### EXPERIMENT 2

A 50 mL glass-lined autoclave, equipped with a PTFE-coated magnetic stirrer bar, was charged with 3 mL trifluoroacetic acid, and the desired quantities of palladium acetate, a second oxidant and optionally sodium nitrite. The reactor was purged three times with methane at 30 atm. The autoclave was then charged with methane (54 atm partial pressure) and optionally oxygen (1 atm partial pressure), and then heated in an oil bath held at 80° C. with constant stirring. After 10 hours, the reaction was quenched by cooling in an ice bath and releasing the pressure.

The product identities were determined using GC-MS and NMR spectroscopy, and quantified by GC, and the quantity of Pd(II) remaining in solution was determined by gravimetric analysis after precipitation.

### COMPARATIVE EXAMPLES 1 TO 7

Conversions of methane to methyl trifluoroacetate in the presence of trifluoroacetic acid using a palladium catalyst were evaluated according to the procedure outlined in Experiment 1. These Examples are not in accordance with the present invention as there was no source of nitrous oxide.

The results of methane oxidation experiments in the presence of different second oxidants are shown in Table 1. The results show the surprisingly superior yields of methyl trifluoroacetate achieved using para-quinone as the second oxidant compared to other oxidants.

Only stoichiometric conversions of methane were achievable, as no oxygen or other first oxidant were used in the reaction to re-oxidise the second oxidant of the catalyst, and hence the palladium.

### COMPARATIVE EXAMPLES 8 TO 11

The procedure of Experiment 2 was used. No sodium nitrite was added. Results are shown in Table 2. These are not examples according to the present invention, as there was no source of nitrous oxide.

### COMPARATIVE EXAMPLES 12 TO 15

Conversions of methane to methyl trifluoroacetate using a palladium catalyst, para-quinone as the second oxidant were evaluated in the absence of sodium nitrite following the procedure of Experiment 2. These Examples are not in accordance with the present invention as there was no source of nitrous oxide. The results are shown in Table 3.

### EXAMPLES 16 TO 20

The procedure of Experiment 2 was followed, using para-quinone as a second oxidant and sodium nitrite as a source of nitrous oxide. These Examples are in accordance with the present invention. Results are shown in Table 4.

TABLE 1

| | | | | CH₃COOCH₃ |
|---|---|---|---|---|
| | Pd(OAc)₂ | Second | Second Oxidant | Yield |
| Example | (mmol) | Oxidant | Quantity (mmol) | (%) ^{a} |
| 1 | 0.10 | — | — | 70 |
| 2 | 0.05 | Cu(OAc)₂ | 0.5 | 80 |
| 3 | 0.05 | FeCl₃ | 0.5 | 60 |
| 4 | 0.05 | K₂S₂O₈ | 0.5 | 120 |
| 5 | 0.05 | p-Quinone | 0.5 | 240 |
| 6 | 0.05 | LiNO₃ | 0.5 | 140 |
| 7 | 0.05 | H₂O₂ | 0.88 | 180 |

^{a} Based on Pd(OAc)₂

TABLE 2

| | | | | CH₃COOCH₃ |
|---|---|---|---|---|
| | Pd(OAc)₂ | Second | Second Oxidant | Yield |
| Example | (μmol) | Oxidant | Quantity (μmol) | (%) |
| 8 | 10 | NHPI ^{b} | 20 | 27 |
| 9 | 10 | CoCl₂ | 20 | 29 |
| 10 | 10 | VOSO₄ | 20 | 17 |
| 11 | 10 | 5% Ru/C ^{c} | 5 mg | 12 |

^{b} N-Hydroxypthalimide
^{c} A heterogeneous catalyst of 5 wt % Ruthenium supported on carbon.

TABLE 3

| | Pd(OAc)₂ | p-Quinone | NaNO₂ | O₂ | CF₃COOCH₃ | Pd²⁺ Remaining |
|---|---|---|---|---|---|---|
| Example | (μmol) | (μmol) | (μmol) | (atm) | Yield (μmol) | (%) ^{d} |
| 12 | 10 | 0 | 0 | 0 | 9.5 | b.d. ^{e} |
| 13 | 10 | 20 | 0 | 0 | 30 | b.d. ^{e} |
| 14 | 10 | 50 | 0 | 0 | 55 | b.d. ^{e} |
| 15 | 10 | 20 | 0 | 1 | 34 | 15 |
| 16 | 10 | 50 | 0 | 1 | 67 | 27 |
| 17 | 10 | 20 | 20 | 1 | 69 | 98 |
| 18 | 10 | 50 | 100 | 1 | 70 | 95 |
| 19 | 5 | 20 | 20 | 1 | 32 | 95 |
| 20 | 20 | 20 | 20 | 1 | 106 | 54 |

^{d} Percentage of palladium remaining in solution at the end of the reaction.
^{e} below detection.

The Examples demonstrate that the presence of a source of nitrous oxide can significantly increase the concentration of the oxidised form of redox active metal centres, which can result in prolonged catalyst lifetime. The results also demonstrate that significantly improved yields of oxygenated hydrocarbon products are achievable using a combination of para-quinone as the oxidant and a source of nitrous oxide.

1-10. (canceled)

11. A catalyst for the oxidation of an alkane to an oxygenated hydrocarbon in the presence of oxygen as a first oxidant, which catalyst comprises a redox active metal centre, an acid, a second oxidant, and a source of nitrous oxide, characterised by the acid being selected from trifluoroacetic acid, oleum, methyl sulphonic acid and trifluoromethyl sulphonic acid.

12. A catalyst as claimed in claim 11, in which the redox active metal centre undergoes a two electron redox cycle when in use.

13. A catalyst as claimed in claim 11, in which the redox active metal centre is selected from V, Fe, Co, Ni, Cu, Zn, Pd, Ag, In, Sn, Sb, Te, Pt, Au, Pb, Bi, Ga, Ge, As, Rh, Ir, Os and Ru.

14. A catalyst as claimed in claim 13, in which the redox active metal centre is selected from V, Fe, Co, Ni, Cu, Rh, Pd and Pt.

15. A catalyst as claimed in claim 14, in which the redox active metal centre is Ni, Rh, Pd or Pt.

16. A catalyst as claimed in claim 11, in which the second oxidant is selected from a second redox active metal centre, a peroxide, a peracid, a quinone and a quinone derivative.

17. A catalyst as claimed in claim 16, in which the second oxidant is para-quinone or a derivative thereof.

18. A catalyst as claimed in claim 11, in which the acid is trifluoroacetic acid.

19. A catalyst as claimed in claim 11, in which the source of nitrous oxide is selected from one or more of nitrous oxide itself, other oxides of nitrogen such as NO₂, N₂O₃, N₂O₄, and N₂O₅, NO₂− (nitrite) salts, and salts comprising NO+ (nitrosonium) cations.

20. A catalyst as claimed in claim 19, in which the source of nitrous oxide is a nitrite salt.

21. A process for the oxidation of an alkane to an oxygenated hydrocarbon in the presence of oxygen, which process comprises contacting a hydrocarbon and oxygen with a catalyst comprising a redox active metal centre, an acid, a second oxidant, characterised by the catalyst also comprising a source of nitrous oxide.

22. A process as claimed in claim 21, in which the redox active metal centre undergoes a two electron redox cycle when in use.

23. A process as claimed in claim 21, in which the redox active metal centre of the catalyst is selected from V, Fe, Co, Ni, Cu, Zn, Pd, Ag, In, Sn, Sb, Te, Pt, Au, Pb, Bi, Ga, Ge, As, Rh, Ir, Os and Ru.

24. A process as claimed in claim 23, in which the redox active metal centre is selected from V, Fe, Co, Ni, Cu, Rh, Pd and Pt.

25. A process as claimed in claim 24, in which the redox active metal centre is Ni, Rh, Pd or Pt.

26. A process as claimed in claim 21, in which the second oxidant is selected from a second redox active metal centre, a peroxide, a peracid, a quinone and a quinine derivative.

27. A process as claimed in claim 26, in which the second oxidant is para-quinone or a derivative thereof.

28. A process as claimed in claim 21, in which the acid is a strong Brønsted acid.

29. A process as claimed in claim 28, in which the acid is selected from heteropolyacids, sulphuric acid, oleum, methyl sulphonic acid, trifluoromethyl sulphonic acid, and trifluoroacetic acid.

30. A process as claimed in claim 29, in which the acid is trifluoroacetic acid.

31. A process as claimed in claim 21, in which the source of nitrous oxide is selected from one or more of nitrous oxide itself, other oxides of nitrogen such as NO², N²O³, N²O₄, and N²O₅, NO² (nitrite) salts, and salts comprising NO+ (nitrosonium) cations.

32. A process as claimed in claim 31, in which the source of nitrous oxide is a nitrite salt.

33. A process as claimed in claim 21, in which the alkane is a C1 to C4 alkane.

34. A process as claimed in claim 21, in which the oxygenated hydrocarbon is an alcohol.
